(19) United States
(12) Patent Application Publication
(10) Pub. No.: US 2024/0239830 A1
(43) Pub. Date: Jul. 18, 2024

(54) METHODS OF PREPARING CARBANUCLEOSIDES USING AMIDES

(71) Applicant: GILEAD SCIENCES, INC., Foster City, CA (US)

(72) Inventors: Stacy Bremner, Edmonton (CA); Zhonghu Chen, Taizhou (CN); Jesse W. Li, Foster City, CA (US); Tiago Vieira, Edmonton (CA); Xiaotian Wang, Edmonton (CA); Yineng Wang, Taizhou (CN); Boran Xu; Lu Yu, Taizhou (CN); Shuai Yu, Taizhou (CN); Yaolei Yu, Taizhou (CN)

(21) Appl. No.: 18/286,971

(22) PCT Filed: Apr. 15, 2022
(86) PCT No.: PCT/US2022/071736

§ 371 Date: Oct. 13, 2023

(30) Foreign Application Priority Data
Apr. 16, 2021 (WO) PCT/CN2021/087731

(51) Int. Cl. C07H 15/207; C07D 487/04

(52) U.S. Cl. CPC C07H 15/207; C07D 487/04

(57) ABSTRACT

The present disclosure describes methods of preparing carbanucleosides.

### CROSS-REFERENCE TO RELATED APPLICATIONS

This application claims priority to PCT/CN2021/087731 filed Apr. 16, 2021, entitled METHODS OF PREPARING CARBANUCLEOSIDES USING AMIDES.

### BACKGROUND

The compound 7-((3S,4R,5R)-3,4-bis(benzyloxy)-5-((benzyloxy)methyl)tetrahydrofuran-2-yl)pyrrolo[2,1-f][1,2,4]triazin-4-amine and substituted compounds thereof are important synthetic intermediates (see, for example, WO2016/069825). There continues to be a need for improved methods of preparing such intermediates, and other carbanucleosides.

### BRIEF SUMMARY

In one embodiment, the present disclosure provides a method of preparing a compound of Formula (II-a) or Formula (II-b):

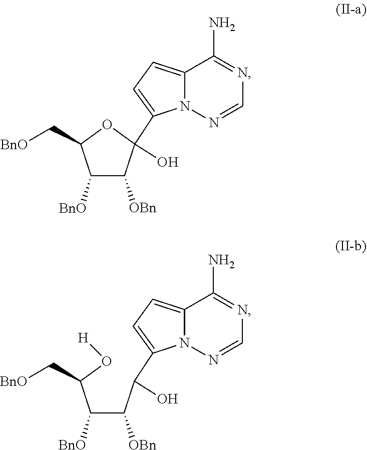 [SMILES: Nc1ncnn2c(C3(O)O[C@H](COCc4ccccc4)[C@@H](OCc4ccccc4)[C@H]3OCc3ccccc3)ccc12 ; Nc1ncnn2c(C(O)[C@H](OCc3ccccc3)[C@H](OCc3ccccc3)[C@H](O)COCc3ccccc3)ccc12]

comprising:
(a) preparing a first input mixture, wherein the first input mixture comprises an amine protecting agent, a first base, a metalating agent, and a compound of Formula (IV):

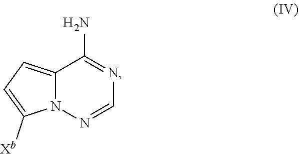

to provide a first output mixture; and
(b) preparing a second input mixture comprising the first output mixture and a compound of Formula (V) to provide a second output mixture comprising the compound of Formula (II-a) or Formula (II-b), wherein the compound of Formula (V) has the structure:

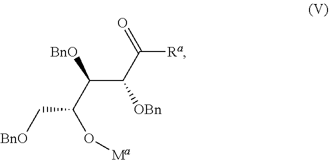

wherein
R^{a} is

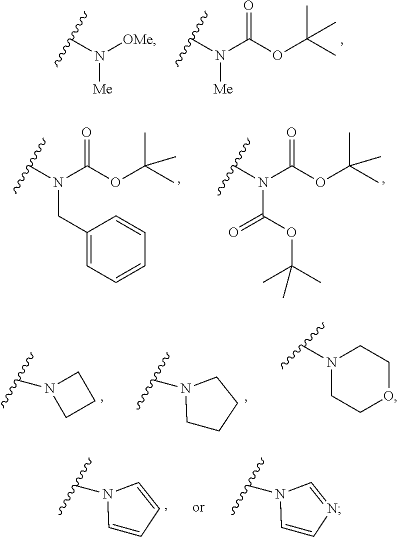

M^{a} is Li or MgX^{a};
X^{a} is Cl, Br, or I; and
X^{a} is Cl, Br, or I.

In another embodiment, the present disclosure provides a method of preparing a compound of Formula (II-a) or Formula (II-b):

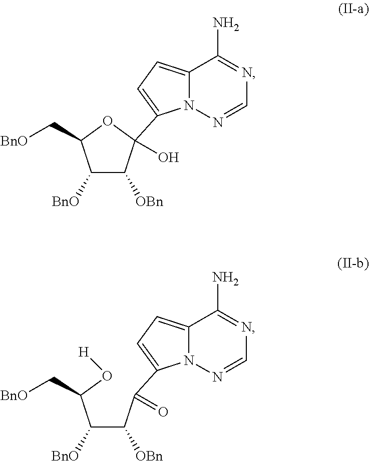 [SMILES: Nc1ncnn2c(C3(O)O[C@H](COCc4ccccc4)[C@@H](OCc4ccccc4)[C@H]3OCc3ccccc3)ccc12 ; Nc1ncnn2c(C(=O)[C@H](OCc3ccccc3)[C@H](OCc3ccccc3)[C@H](O)COCc3ccccc3)ccc12]

comprising:
(a) preparing a first input mixture in a first reactor, wherein the first input mixture comprises an amine protecting agent, a first base, a metalating agent, and a compound of Formula (IV):

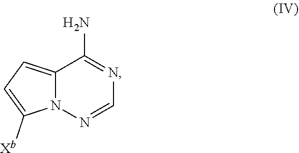

wherein the first reactor provides a first output mixture; and
(b) adding the first output mixture and a compound of Formula (V) to form a second input mixture in a second reactor, wherein the compound of Formula (V) has the structure:

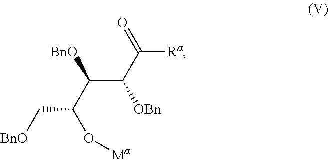

wherein
R^{a} is

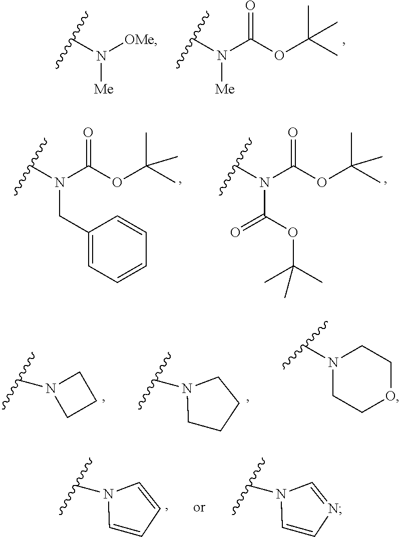

M^{a} is Li or MgX^{a};
X^{a} is Cl, Br, or I;
X^{b} is Cl, Br, or I; and
   the second reactor provides a second output mixture comprising the compound of Formula (II-a) or Formula (II-b).

### DETAILED DESCRIPTION

### I. GENERAL

The present disclosure describes methods of preparing carbanucleosides. The methods described herein can relate to efficient, scalable processes that can be performed at any scale. In some embodiments, the method comprises preparing the compound of Formula (II-a) or Formula (II-b):

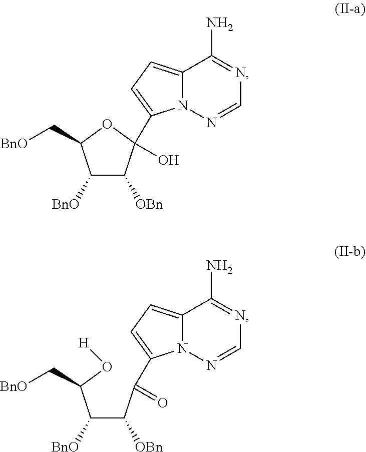 [SMILES: Nc1ncnn2c(C3(O)O[C@H](COCc4ccccc4)[C@@H](OCc4ccccc4)[C@H]3OCc3ccccc3)ccc12 ; Nc1ncnn2c(C(=O)[C@H](OCc3ccccc3)[C@H](OCc3ccccc3)[C@H](O)COCc3ccccc3)ccc12]

from the compound of Formula (V):

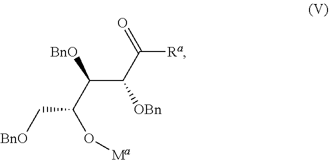

wherein R^{a}, and M^{a} are as defined herein.

### II. DEFINITIONS

“About” when referring to a value includes the stated value +/−10% of the stated value. For example, about 50% includes a range of from 45% to 55%, while about 20 molar equivalents includes a range of from 18 to 22 molar equivalents. Accordingly, when referring to a range, “about” refers to each of the stated values +/−10% of the stated value of each end of the range. For instance, a ratio of from about 1 to about 3 (weight/weight) includes a range of from 0.9 to 3.3.

“Input mixture” as used herein refers to a mixture of one or more reagents and/or solvents that enters a reactor.

“Output mixture” as used herein refers to a mixture of one or more reagents and/or solvents that exits a reactor.

“Reactor” refers to a vessel to which chemicals and reagents are added as an input mixture, and configured so that conversion of the chemicals, reagents, and other dependent variables are performed within the reactor. Each reactor can separately be a round-bottom flask, a batch reactor, a continuous flow reactor, a plug flow reactor, a continuous tubular reactor, a continuous stirred tank reactor, a mixed flow reactor, a semi-batch reactor, or combinations thereof. One or more reactors can be used in the method of the present disclosure. When multiple reactors are present the reactors can be of the same or different types of reactors.

“Catalyst” refers to a chemical reactant that increases the rate of a reaction without itself being consumed.

“Lewis acid” refers to a chemical group capable of accepting an electron pair from a second chemical group capable of donating an electron pair. Lewis acids can be inorganic compounds including, but not limited to, boron salts, such as boron trifluoride, or aluminum salts, such as aluminum trichloride; organic compound salts, such as trimethylsilyl trifluoromethanesulfonate (trimethylsilyl triflate or TMSOTf); or metal complexes containing organic and/or inorganic ligands, such as indium(III) chloride or dichlorodiisopropoxytitanium(IV). Exemplary Lewis acids include, but are not limited to, boron trifluoride diethyl etherate (BF₃·Et₂O), trimethylsilyl trifluoromethanesulfonate (trimethylsilyl triflate or TMSOTf), TiCl₄, SnCl₄, and FeCl₃.

“Bronsted acid,” “Brønsted acid,” or “Brønsted-Lowry acid” refers to an acid capable of donating a proton and forming the conjugate base. Examples of Bronsted acids include, but are not limited to, inorganic acids such as hydrogen chloride, hydrogen bromide, hydrogen iodide, hydrogen tetrafluoroborate, and sulfuric acid; and organic acids, e.g., carboxylic acids such as acetic acid and trifluoroacetic acid (TFA), or sulfonic acids such as p-toluenesulfonic acid and trifluoromethanesulfonic acid. Exemplary Bronsted acids include, but are not limited to, formic acid, acetic acid, dichloroacetic acid, and trifluoroacetic acid.

An “inorganic acid” or “mineral acid” is an acid derived from one or more inorganic compounds. Inorganic acids form hydrogen ions and the conjugate base when dissolved in water. Exemplary inorganic acids include, but are not limited to, hydrochloric acid and phosphoric acid.

An “organic acid” is an organic compound, a chemical compound containing a carbon-hydrogen bond, that has an acidic moiety. Organic acids include, but are not limited to, alkanecarboxylic acids, whose acidity is associated with their carboxyl group —COOH, and arylsulfonic acids, containing the group —SO₂OH. Exemplary organic acids include, but are not limited to, acetic acid and p-toluenesulfonic acid.

“Protecting group” refers to a moiety of a compound that masks or alters the properties of a functional group or the properties of the compound as a whole. The chemical substructure of a protecting group varies widely. One function of a protecting group is to serve as an intermediate in the synthesis of a desired compound. Chemical protecting groups and strategies for protection/deprotection are well known in the art. See also Protective Groups in Organic Chemistry, Peter G. M. Wuts and Theodora W. Greene, 4th Ed., 2006. Protecting groups are often utilized to mask the reactivity of certain functional groups, to assist in the efficiency of desired chemical reactions, e.g., making and breaking chemical bonds in an ordered and planned fashion. “Amine protecting group” refers to a protecting group useful for protecting amines bearing at least one uncharged hydrogen.

A “protecting agent” is a chemical reactant that is capable of effecting attachment of a protecting group. An “amine protecting agent” is a reactant capable of effecting attachment of an amine protecting group onto an amine.

“Metalating agent” is a chemical reactant that is capable of effecting the transfer of an organic ligand from a compound, wherein the ligand has a carbon bound to a metal atom on the compound.

### III. METHODS OF PREPARING

Provided herein are methods of preparing a compound of Formula (II-a) or Formula (II-b) at various scales, such as multigram or kilogram scale. In some embodiments, the present disclosure provides a method of preparing a compound of Formula (II-a) or Formula (II-b):

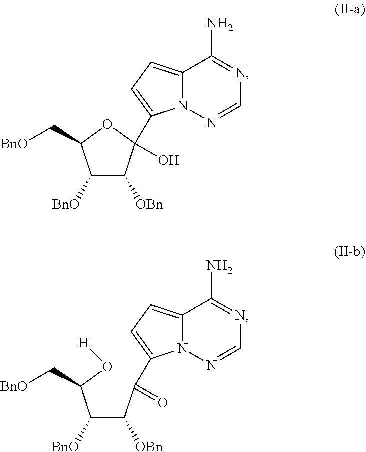 [SMILES: Nc1ncnn2c(C3(O)O[C@H](COCc4ccccc4)[C@@H](OCc4ccccc4)[C@H]3OCc3ccccc3)ccc12 ; Nc1ncnn2c(C(=O)[C@H](OCc3ccccc3)[C@H](OCc3ccccc3)[C@H](O)COCc3ccccc3)ccc12]

comprising:
(a) preparing a first input mixture, wherein the first input mixture comprises an amine protecting agent, a first base, a metalating agent, and a compound of Formula (IV):

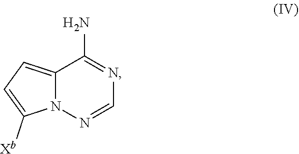

to provide a first output mixture; and
(b) preparing a second input mixture comprising the first output mixture and a compound of Formula (V) to provide a second output mixture comprising the compound of Formula (II-a) or Formula (II-b), wherein the compound of Formula (V) has the structure:

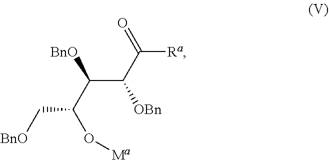

wherein
R^{a} is

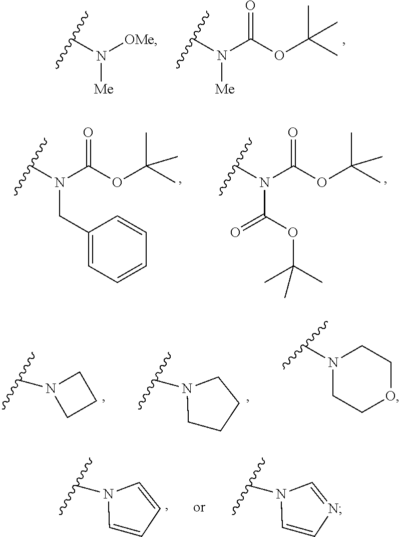

M^{a} is Li or MgX^{a};
X^{a} is Cl, Br, or I; and
X^{b} is Cl, Br, or I.

In some embodiments, the present disclosure provides a method of preparing a compound of Formula (II-a) or Formula (II-b):

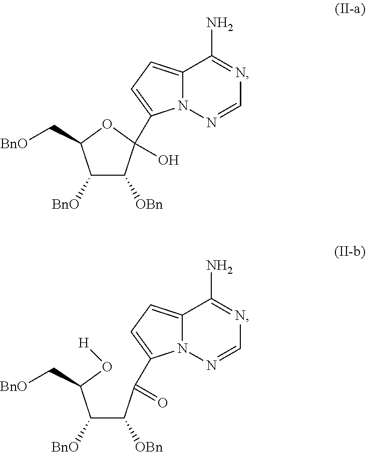 [SMILES: Nc1ncnn2c(C3(O)O[C@H](COCc4ccccc4)[C@@H](OCc4ccccc4)[C@H]3OCc3ccccc3)ccc12 ; Nc1ncnn2c(C(=O)[C@H](OCc3ccccc3)[C@H](OCc3ccccc3)[C@H](O)COCc3ccccc3)ccc12]

comprising:
(a) preparing a first input mixture in a first reactor, wherein the first input mixture comprises an amine protecting agent, a first base, a metalating agent, and a compound of Formula (IV):

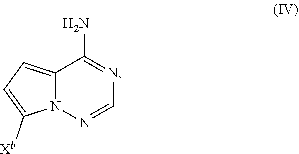

wherein the first reactor provides a first output mixture; and
(b) adding the first output mixture and a compound of Formula (V) to form a second input mixture in a second reactor, wherein the compound of Formula (V) has the structure:

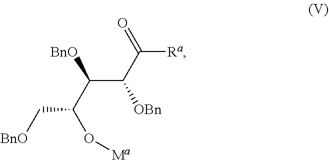

wherein
R^{a} is

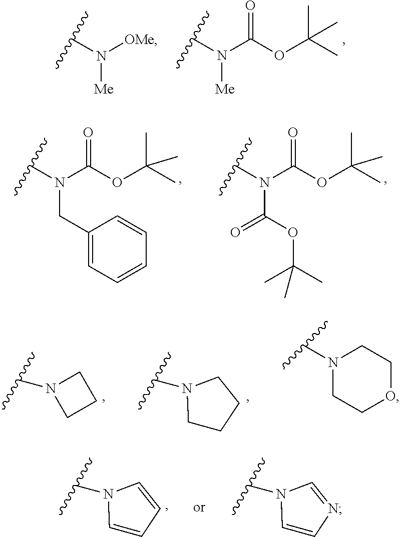

M^{a} is Li or MgX^{a};
X^{a} is Cl, Br, or I;
X^{b} is Cl, Br, or I; and
   the second reactor provides a second output mixture comprising the compound of Formula (II-a) or Formula (II-b).

In some embodiments, the present disclosure provides a method of preparing a compound of Formula (II-a) or Formula (II-b):

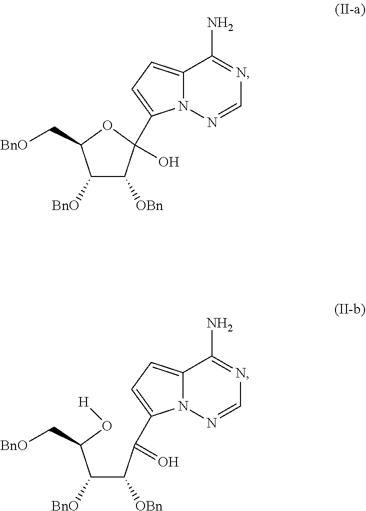 [SMILES: Nc1ncnn2c(C3(O)O[C@H](COCc4ccccc4)[C@@H](OCc4ccccc4)[C@H]3OCc3ccccc3)ccc12 ; Nc1ncnn2c(C(=O)[C@H](OCc3ccccc3)[C@H](OCc3ccccc3)[C@H](O)COCc3ccccc3)ccc12]

comprising:
   (a) reacting a first input mixture to provide a first output mixture; and
   (b) reacting the first output mixture with a compound of Formula (V) to provide the compound of Formula (II-a) or Formula (II-b);
   wherein the first input mixture comprises an amine protecting agent, a first base, a metalating agent, and a compound of Formula (IV):

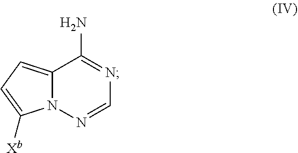

and
wherein the compound of Formula (V) is:

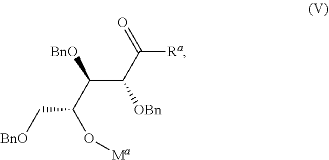

wherein
R^{a} is

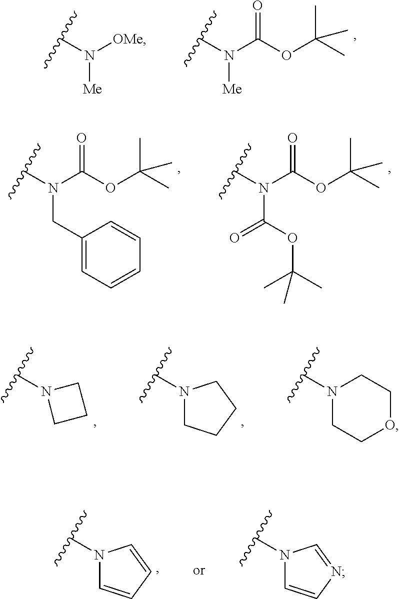

M^{a} is Li or MgX^{a};
X^{a} is Cl, Br, or I; and
X^{b} is Cl, Br, or I.

In some embodiments, the method for preparing the compound of Formula (II-a) or Formula II-b):

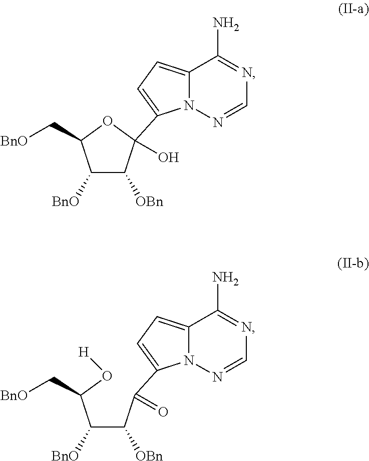 [SMILES: Nc1ncnn2c(C3(O)O[C@H](COCc4ccccc4)[C@@H](OCc4ccccc4)[C@H]3OCc3ccccc3)ccc12 ; Nc1ncnn2c(C(=O)[C@H](OCc3ccccc3)[C@H](OCc3ccccc3)[C@H](O)COCc3ccccc3)ccc12]

comprises:
(a) preparing the first input mixture in the first reactor, wherein the first input mixture comprises the amine protecting agent, the first base, the metalating agent, and the compound of Formula (IV):

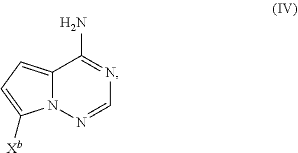

wherein the first reactor provides the first output mixture; and
(b) adding the first output mixture and the compound of Formula (V) to form the second input mixture in the second reactor, wherein the compound of Formula (V) has the structure:

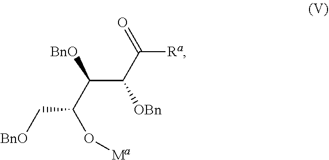

wherein
R^{a} is

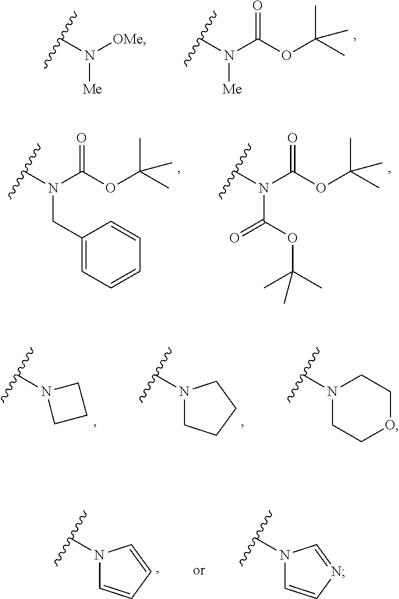

M^{a} is Li or MgX^{a};
X^{a} is Cl, Br, or I;
X^{b} is Cl, Br, or I;
the first base is R¹MgX¹ or R¹Li;
R¹ is methyl, ethyl, n-propyl, isopropyl, n-butyl, tert-butyl, or phenyl;
X¹ is Cl, Br, or I; and
the second reactor provides a second output mixture comprising the compound of Formula (II-a) or Formula (II-b),
with the proviso that when R^{a} is

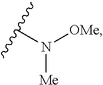 [SMILES: *N(C)OC]

M^{a} is MgCl, and R¹ is methyl, then X¹ is Cl or I.

In some embodiments, the method for preparing the compound of Formula (II-a) or Formula II-b):

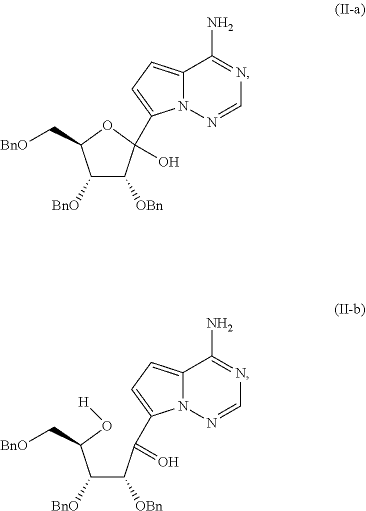 [SMILES: Nc1ncnn2c(C3(O)O[C@H](COCc4ccccc4)[C@@H](OCc4ccccc4)[C@H]3OCc3ccccc3)ccc12 ; Nc1ncnn2c(C(=O)[C@H](OCc3ccccc3)[C@H](OCc3ccccc3)[C@H](O)COCc3ccccc3)ccc12]

comprises:
(a) preparing the first input mixture in the first reactor, wherein the first input mixture comprises the amine protecting agent, the first base, the metalating agent, and the compound of Formula (IV):

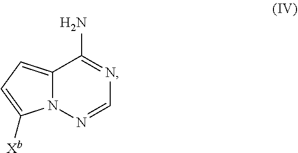

wherein the first reactor provides the first output mixture; and
(b) adding the first output mixture and the compound of Formula (V) to form the second input mixture in the second reactor, wherein the compound of Formula (V) has the structure:

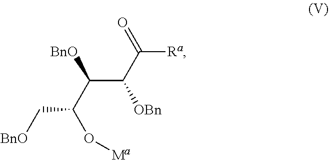

wherein
R^{a} is

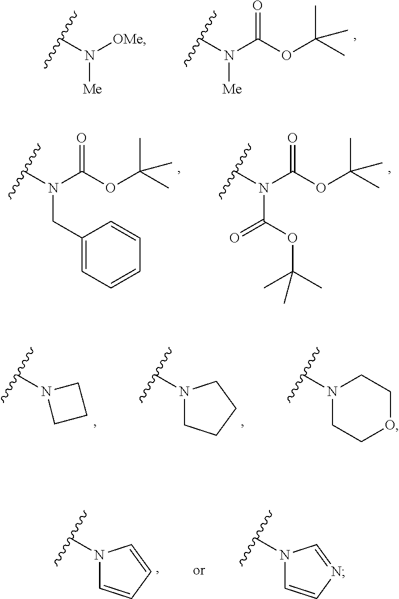

M^{a} is Li or MgX^{a};
X^{a} is Cl, Br, or I;
X^{b} is Cl, Br, or I;
the metalating agent is R²MgX² or R²Li;
R² is methyl, ethyl, n-propyl, isopropyl, n-butyl, tert-butyl, or phenyl;
X² is Cl, Br, or I; and
the second reactor provides a second output mixture comprising the compound of Formula (II-a) or Formula (II-b).

In some embodiments, the method for preparing the compound of Formula (II-a) or Formula II-b):

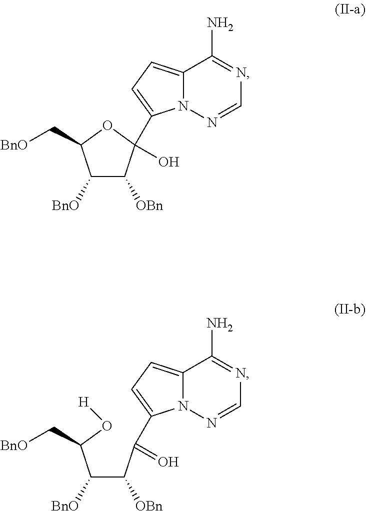 [SMILES: Nc1ncnn2c(C3(O)O[C@H](COCc4ccccc4)[C@@H](OCc4ccccc4)[C@H]3OCc3ccccc3)ccc12 ; Nc1ncnn2c(C(=O)[C@H](OCc3ccccc3)[C@H](OCc3ccccc3)[C@H](O)COCc3ccccc3)ccc12]

comprising:
(a) preparing the first input mixture in the first reactor, wherein the first input mixture comprises the amine protecting agent, the first base, the metalating agent, and the compound of Formula (IV):

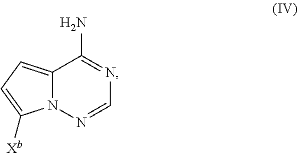

wherein the first reactor provides the first output mixture; and
(b) adding the first output mixture and the compound of Formula (V) to form the second input mixture in the second reactor, wherein the compound of Formula (V) has the structure:

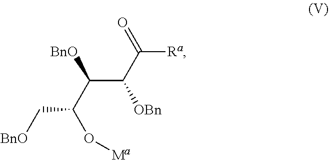

wherein
R^{a} is

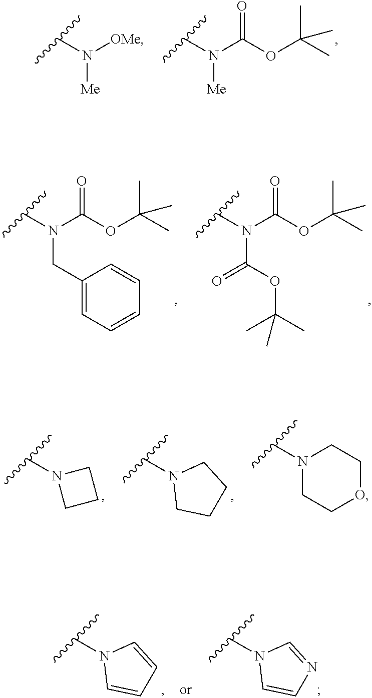

M^{a} is Li or MgX^{a};
X^{a} is Cl, Br, or I;
X^{b} is Cl, Br, or I;
the first base is R¹MgX¹ or R¹Li;
R¹ is methyl, ethyl, n-propyl, isopropyl, n-butyl, tert-butyl, or phenyl;
X¹ is Cl, Br, or I;
the metalating agent is R²MgX² or R²Li;
R² is methyl, ethyl, n-propyl, isopropyl, n-butyl, tert-butyl, or phenyl;
X² is Cl, Br, or I; and
the second reactor provides a second output mixture comprising the compound of Formula (II-a) or Formula (II-b),
with the proviso that when R^{a} is

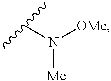 [SMILES: *N(C)OC]

M^{a} is MgCl, and R¹ is methyl, then X¹ is Cl or I.

In some embodiments, X^{a} is Cl, Br, or I. In some embodiments, X^{a} is Br or I. In some embodiments, X^{a} is Cl. In some embodiments, X^{a} is Br. In some embodiments, X^{a} is I.

In some embodiments, M^{a} is Li or MgX^{a}. In some embodiments, M^{a} is Li. In some embodiments, M^{a} is MgX^{a}. In some embodiments, M^{a} is MgCl. In some embodiments, M^{a} is MgBr. In some embodiments, M^{a} is MgI.

In some embodiments, X^{b} is Cl, Br, or I. In some embodiments, X^{b} is Br or I. In some embodiments, X^{b} is Cl. In some embodiments, X^{b} is Br. In some embodiments, X^{b} is I.

In some embodiments, the compound of Formula (IV) has the structure:

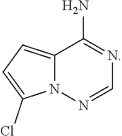 [SMILES: Nc1ncnn2c(Cl)ccc12]

In some embodiments, the compound of Formula (IV) has the structure:

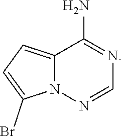 [SMILES: Nc1ncnn2c(Br)ccc12]

In some embodiments, the compound of Formula (IV) has the structure:

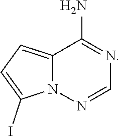 [SMILES: Nc1ncnn2c(I)ccc12]

In some embodiments, R^{a} is

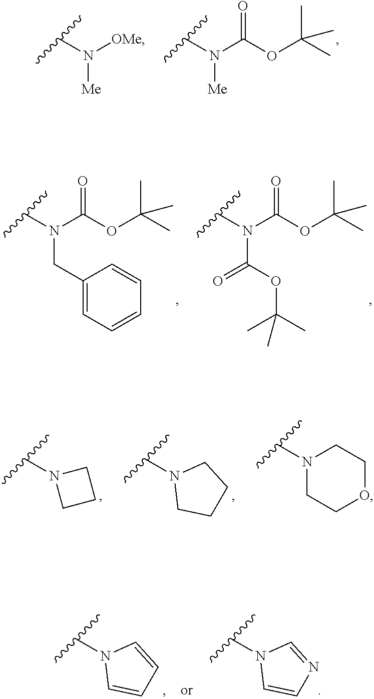

In some embodiments, R^{a} is

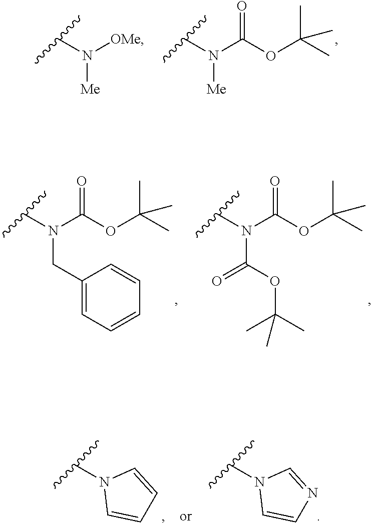

In some embodiments, R^{a} is

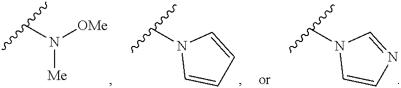 [SMILES: *N(C)OC ; *n1cccc1 ; *n1ccnc1]

In some embodiments, R^{a} is

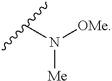 [SMILES: *N(C)OC]

In some embodiments, the compound of Formula (V) has the structure:

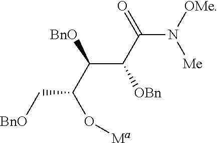

In some embodiments, the compound of Formula (V) has the structure:

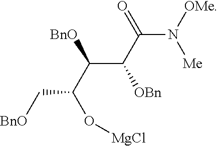

In some embodiments, the compound of Formula (V) has the structure:

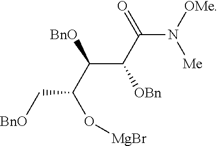

In some embodiments, the compound of Formula (V) has the structure:

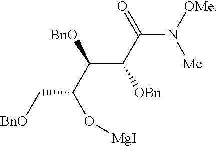

Any suitable amine protecting agent known in the art can be used in preparing the compound of Formula (II-a) or Formula (II-b). In some embodiments, the amine protecting agent is an anhydride, a silyl halide, or a silyl trifluoromethanesulfonate. Suitable anhydrides include, but are not limited to, trifluoroacetic anhydride and di(tert-butyl) dicarbonate. Silyl halides include, but are not limited to, trimethylsilyl halide (TMS-X⁴), triethylsilyl halide (TES-X⁴), triisopropylsilyl halide (TIPS-X⁴), tert-butyldimethylsilyl halide (TBDMS-X⁴), tert-butyldiphenylsilyl halide (TBDPS-X⁴), triphenylsilyl halide (TPS-X⁴), 1,2-bis(halodimethylsilyl)ethane (X⁴Me₂SiCH₂—CH₂SiMe₂X⁴), wherein X⁴ is Cl, Br, or I. Silyl trifluoromethanesulfonates include, but are not limited to, trimethylsilyl trifluoromethanesulfonate (TMSOTf), triethylsilyl trifluoromethanesulfonate (TESOTf), triisopropylsilyl trifluoromethanesulfonate, tert-butyldimethylsilyl trifluoromethanesulfonate (TBDMSOTf), tert-butyldiphenylsilyl trifluoromethanesulfonate (TBDPSOTf), and triphenylsilyl trifluoromethanesulfonate. In some embodiments, the amine protecting agent is trifluoroacetic anhydride, di(tert-butyl) dicarbonate, trimethylsilyl chloride (TMSCl), triethylsilyl chloride (TESCl), triisopropylsilyl chloride, tert-butyldimethylsilyl chloride (TBDMSCl), tert-butyldiphenylsilyl chloride (TBDPSCl), triphenylsilyl chloride, or 1,2-bis(chlorodimethylsilyl)ethane. In some embodiments, the amine protecting agent is trimethylsilyl chloride (TMSCl).

Any suitable first base capable of deprotonating the compound of Formula (IV) can be used in preparing the compound of Formula (II-a) or Formula (II-b). In some embodiments, the first base is a Grignard reagent such as an alkylmagnesium halide optionally complexed with a lithium halide, for example, iPrMgCl or iPrMgCl—LiCl; an alkyllithium reagent; an aryllithium reagent; or an inorganic hydride, such as sodium hydride or potassium hydride. In some embodiments, the first base is R¹MgX¹ or R¹Li; R¹ is methyl, ethyl, n-propyl, isopropyl, n-butyl, tert-butyl, or phenyl; and X¹ is Cl, Br, or I.

In some embodiments, the first base is R¹MgX¹ or R¹Li; R¹ is methyl, ethyl, n-propyl, isopropyl, n-butyl, tert-butyl, or phenyl; and X¹ is Cl, Br, or I, with the proviso that when R¹ is methyl, then X¹ is Cl or I. In some embodiments, the first base is R¹MgX¹ or R¹Li; R¹ is ethyl, n-propyl, isopropyl, n-butyl, tert-butyl, or phenyl; and X¹ is Cl, Br, or I. In some embodiments, the first base is R¹MgX¹ or R¹Li; R¹ is methyl, ethyl, n-propyl, isopropyl, n-butyl, tert-butyl, or phenyl; and X¹ is Cl or I. In some embodiments, the first base is R¹MgX¹ or R¹Li; R¹ is ethyl, n-propyl, isopropyl, n-butyl, tert-butyl, or phenyl; and X¹ is Cl or I.

In some embodiments, the first base is R¹MgX¹; R¹ is methyl, ethyl, n-propyl, isopropyl, n-butyl, tert-butyl, or phenyl; and X¹ is Cl, Br, or I, with the proviso that when R¹ is methyl, then X¹ is Cl or I. In some embodiments, the first base is R¹MgX¹; R¹ is ethyl, n-propyl, isopropyl, n-butyl, tert-butyl, or phenyl; and X¹ is Cl, Br, or I. In some embodiments, the first base is R¹MgX¹; R¹ is methyl, ethyl, n-propyl, isopropyl, n-butyl, tert-butyl, or phenyl; and X¹ is Cl or I. In some embodiments, the first base is R¹MgX¹; R¹ is ethyl, n-propyl, isopropyl, n-butyl, tert-butyl, or phenyl; and X¹ is Cl or I.

In some embodiments, the first base is R¹MgX¹. In some embodiments, R¹ is isopropyl or phenyl. In some embodiments, R¹ is isopropyl. In some embodiments, R¹ is phenyl. In some embodiments, X¹ is Cl. In some embodiments, the first base is iPrMgCl or PhMgCl. In some embodiments, the first base is iPrMgCl. In some embodiments, the first base is PhMgCl.

Any suitable metalating agent capable of effecting transmetallation of the compound of Formula (IV) can be used in preparing the compound of Formula (II-a) or Formula (II-b). For example, the metalating agent is a Grignard reagent such as an alkylmagnesium halide optionally complexed with a lithium halide, for example, iPrMgCl or iPrMgCl—LiCl; an alkyllithium reagent; or an aryllithium reagent. In some embodiments, the metalating agent is R²MgX² or R²Li; R² is methyl, ethyl, n-propyl, isopropyl, n-butyl, tert-butyl, or phenyl; and X² is Cl, Br, or I. In some embodiments, the metalating agent is R²MgX². In some embodiments, R² is isopropyl or phenyl. In some embodiments, R² is isopropyl. In some embodiments, R² is phenyl. In some embodiments, X² is Cl. In some embodiments, the metalating agent is iPrMgCl or PhMgCl. In some embodiments, the metalating agent is iPrMgCl. In some embodiments, the metalating agent is PhMgCl.

In some embodiments, the first base and metalating agent are each alkyllithium reagents. In some embodiments, one of the first base and metalating agent is an alkyllithium reagent, and the other is a Grignard reagent. In some embodiments, the first base and metalating agent are each Grignard reagents. In some embodiments, the first base is PhMgCl; and the metalating agent is iPrMgCl. In some embodiments, the first base is PhMgCl; and the metalating agent is iPrMgCl—LiCl. In some embodiments, the first base is iPrMgCl; and the metalating agent is PhMgCl. In some embodiments, the first base is iPrMgCl; and the metalating agent is iPrMgCl. In some embodiments, the first base is iPrMgCl—LiCl; and the metalating agent is iPrMgCl—LiCl.

In some embodiments, the first base is PhMgCl; the metalating agent is iPrMgCl; and M^{a} is MgCl. In some embodiments, the first base is PhMgCl; the metalating agent is iPrMgCl—LiCl; and M^{a} is MgCl. In some embodiments, the first base is iPrMgCl; the metalating agent is PhMgCl; and M^{a} is MgCl. In some embodiments, the first base is iPrMgCl; the metalating agent is iPrMgCl; and M^{a} is MgCl. In some embodiments, the first base is iPrMgCl—LiCl; the metalating agent is iPrMgCl—LiCl; and M^{a} is MgCl.

In some embodiments, the amine protecting agent is trimethylsilyl chloride (TMSCl); the first base is PhMgCl; the metalating agent is iPrMgCl; and M^{a} is MgCl. In some embodiments, the amine protecting agent is triethylsilyl chloride (TESCl); the first base is PhMgCl; the metalating agent is iPrMgCl; and M^{a} is MgCl. In some embodiments, the amine protecting agent is triisopropylsilyl chloride, tert-butyldimethylsilyl chloride (TBDMSCl), tert-butyldiphenylsilyl chloride (TBDPSCl), triphenylsilyl chloride, or 1,2-bis(chlorodimethylsilyl)ethane; the first base is PhMgCl; the metalating agent is iPrMgCl; and M^{a} is MgCl.

In some embodiments, the first input mixture further comprises a first solvent. In some embodiments, the first output mixture further comprises a first solvent. In some embodiments, a first solvent is added to the first reactor. In some embodiments, a first solvent is added to the second reactor. Any suitable solvent can be used as the first solvent in preparing the compound of Formula (II-a) or Formula (II-b). Suitable solvents include, but are not limited to, ether solvents, such as tetrahydrofuran, 2-methyltetrahydrofuran, methyl tert-butyl ether, and cyclopentyl methyl ether; hydrocarbon solvents, such as toluene and n-heptane; and halogenated solvents, such as 1,2-dichloroethane, chloroform, and chlorobenzene. In some embodiments, the first input mixture further comprises a first solvent that is tetrahydrofuran (THF), 2-methyltetrahydrofuran, methyl tert-butyl ether, cyclopentyl methyl ether, toluene, n-heptane, 1,2-dichloroethane, chloroform, or chlorobenzene, or a combination thereof. In some embodiments, the first solvent is tetrahydrofuran (THF).

Any suitable reactor or combination of reactors known in the art can be used to prepare the compound of Formula (II-a) or Formula (II-b). Exemplary reactors that can be used to prepare the compound of Formula (II-a) or Formula (II-b) include, but are not limited to, batch reactors, continuous flow reactors, plug flow reactors, continuous tubular reactors, continuous stirred tank reactors, mixed flow reactors, semi-batch reactors, or combinations thereof. In some embodiments, one reactor is used. In some embodiments, two reactors are used. In some embodiments, three reactors are used.

In some embodiments, the first reactor and the second are different reactors. In some embodiments, the first reactor and the second reactor are the same type of reactor. In some embodiments, the first reactor and the second reactor are different types of reactors. In some embodiments, the first reactor and the second reactor are a single reactor. In some embodiments, the single reactor is a continuous flow reactor, a plug flow reactor, a continuous tubular reactor, or a mixed flow reactor. In some embodiments, the first reactor is a first reaction zone in the single reactor and the second reactor is a second reaction zone in the single reactor.

In some embodiments, one reactor having a first reaction zone and a second reaction zone is used to prepare a compound of Formula (II-a) or Formula (II-b). The first input mixture can be prepared in the first reaction zone of the reactor at a first set of reaction conditions, which includes a first temperature and a first pressure, for a first amount of time. The first input mixture can react to provide a first output mixture as the mixture moves from the first reaction zone to the second reaction zone. The compound of Formula (V) can be added into the second reaction zone of the reactor at a second set of reaction conditions, which includes a second temperature and a second pressure, for a second amount of time. In some embodiments, the one reactor having a first reaction zone and a second reaction zone is a plug flow reactor. In some embodiments, the one reactor having a first reaction zone and a second reaction zone is a continuous tubular reactor. In some embodiments, the one reactor having a first reaction zone and a second reaction zone comprises a recycle loop. In some embodiments, the first input mixture and the compound of Formula (V) are added separately. In some embodiments, the first input mixture is added to the first reaction zone and the compound of Formula (V) is added to the second reaction zone. In some embodiments, the first input mixture and the compound of Formula (V) are added simultaneously to the first reaction zone.

In some embodiments, one reactor having one reaction zone is used to prepare a compound of Formula (II-a) or Formula (II-b). The first input mixture and the compound of Formula (V) can be added into the one reaction zone at a first set of reaction conditions, which includes a first temperature and a first pressure, for a first amount of time. Then, the one reaction zone of the one reactor can be transitioned to a second set of reaction conditions, which includes a second temperature and a second pressure, for a second amount of time. In some embodiments, the one reactor having one reaction zone is a batch reactor. In some embodiments, the first input mixture is added to the one reaction zone at a first set of reaction conditions, then the compound of Formula (V) is added to the one reaction zone, and the one reactor is transitioned to a second set of reaction conditions. In some embodiments, the one reactor having one reaction zone is a semi-batch reactor. In some embodiments, the first input mixture and the compound of Formula (V) are added to the one reaction zone at a temperature from about −20° C. to about 20° C., at a pressure from about 0.1 bar to about 10 bar, for an amount of time from about 1 hour to about 24 hours to produce a compound of Formula (II-a) or Formula (II-b).

In some embodiments, two reactors including a first reactor and a second reactor are used to prepare a compound of Formula (II-a) or Formula (II-b). The first reactor can operate at a first set of reaction conditions including a first temperature and a first pressure. The second reactor can operate at a second set of reaction conditions including a second temperature and a second pressure. In some embodiments, the first reactor and the second reactor are the same type of reactor. In some embodiments, the first reactor and/or second reactor are batch reactors. In some embodiments, the first reactor and/or the second reactor are different types of reactor. In some embodiments, the first reactor and/or second reactor are semi-batch reactors. In some embodiments, the first reactor and second reactor are continuous stirred tank reactors.

Any suitable temperature can be used in the first reactor for preparing the compound of Formula (II-a) or Formula (II-b). The first reactor is maintained at a suitable first temperature to provide the first output mixture in an appropriate time and yield. In some embodiments, the first reactor is maintained at a first temperature of from about −78° C. to about 20° C. In some embodiments, the first reactor is cooled to a first temperature of from about −20° C. to about 0° C. In some embodiments, the first reactor is cooled to a first temperature of from about −20° C. to about −5° C. In some embodiments, the first reactor is cooled to a first temperature of from about −20° C. to about −10° C. In some embodiments, the first reactor is cooled to a first temperature of about −20° C.

The method of preparing the compound of Formula (II-a) or Formula (II-b) can be performed at any suitable pressure. For example, the first reactor can have a first pressure. A suitable first pressure can be less than atmospheric pressure, atmospheric pressure, or greater than atmospheric pressure. Other suitable first pressures can be, but are not limited to, 0.1 to 10 bar, 0.2 to 9 bar, 0.3 to 8 bar, 0.4 to 7 bar, 0.5 to 6 bar, 0.6 to 5 bar, 0.7 to 4 bar, 0.8 to 3 bar, 0.9 to 2 bar, or about 1 bar. In some embodiments, the first pressure can be atmospheric pressure. In some embodiments, the first pressure can be about 1 bar.

The method of preparing the compound of Formula (II-a) or Formula (II-b) can be performed for any suitable period of time. For example, a first period of time for preparing the compound of Formula (II-a) or Formula (II-b) can be, but is not limited to, 1 to 600 minutes, 30 to 600 minutes, 60 to 600 minutes, 60 to 300 minutes, 60 to 240 minutes, 60 to 180 minutes, 90 to 150 minutes, or about 120 minutes. In some embodiments, a first period of time for preparing the compound of Formula (II-a) or Formula (II-b) can be about 120 minutes. In some embodiments, a first period of time for preparing the compound of Formula (II-a) or Formula (II-b) can be about 90 minutes.

In some embodiments, the second input mixture further comprises a second solvent. In some embodiments, the second output mixture further comprises a second solvent. In some embodiments, a second solvent is added to the second reactor. In some embodiments, the second solvent is the same as the first solvent. In some embodiments, the second solvent is different from the first solvent. Any suitable solvent can be used as the second solvent in preparing the compound of Formula (II-a) or Formula (II-b). Suitable solvents include, but are not limited to, ether solvents, such as tetrahydrofuran, 2-methyltetrahydrofuran, methyl tert-butyl ether, and cyclopentyl methyl ether; hydrocarbon solvents, such as toluene and n-heptane; and halogenated solvents, such as 1,2-dichloroethane, chloroform, and chlorobenzene. In some embodiments, the second input mixture further comprises a second solvent that is tetrahydrofuran (THF), 2-methyltetrahydrofuran, methyl tert-butyl ether, cyclopentyl methyl ether, toluene, n-heptane, 1,2-dichloroethane, chloroform, or chlorobenzene, or a combination thereof. In some embodiments, the second solvent is tetrahydrofuran (THF).

Any suitable temperature can be used in the second reactor for preparing the compound of Formula (II-a) or Formula (II-b). The second reactor is maintained at a suitable temperature to provide the second output mixture comprising the compound of Formula (II-a) or Formula (II-b) in an appropriate time and yield. In some embodiments, the second reactor is maintained at a temperature of from about −20° C. to about 40° C. In some embodiments, the second reactor is maintained at a temperature of from about 10° C. to about 30° C. In some embodiments, the second reactor is maintained at a temperature of about 20° C.

The method of preparing the compound of Formula (II-a) or Formula (II-b) can be performed at any suitable pressure. For example, the second reactor can have a second pressure. A suitable second pressure can be less than atmospheric pressure, atmospheric pressure, or greater than atmospheric pressure. Other suitable first pressures can be, but are not limited to, 0.1 to 10 bar, 0.2 to 9 bar, 0.3 to 8 bar, 0.4 to 7 bar, 0.5 to 6 bar, 0.6 to 5 bar, 0.7 to 4 bar, 0.8 to 3 bar, 0.9 to 2 bar, or about 1 bar. In some embodiments, the first pressure can be atmospheric pressure. In some embodiments, the first pressure can be about 1 bar.

The method of preparing the compound of Formula (II-a) or Formula (II-b) can be performed for any suitable period of time. For example, a second period of time for preparing the compound of Formula (II-a) or Formula (II-b) can be, but is not limited to, 1 to 50 hours, 1 to 48 hours, 1 to 40 hours, 1 to 30 hours, 1 to 24 hours, 2 to 12 hours, 4 to 12 hours, 6 to 10 hours, 6 to 24 hours, 10 to 20 hours, or 12 to 18 hours. In some embodiments, a second period of time for preparing the compound of Formula (II-a) or Formula (II-b) can be about 8 hours. In some embodiments, a second period of time for preparing the compound of Formula (II-a) or Formula (II-b) can be from 12 to 18 hours.

The compound of Formula (II-a) or Formula (II-b) can be isolated by any suitable method known in the art, including concentration, extraction, trituration, crystallization, and/or chromatography.

In some embodiments, the method further comprises combining the second output mixture and an acid. In some embodiments, the acid comprises a Bronsted acid. In some embodiments, the acid comprises an organic acid or a mineral acid, or combinations thereof. In some embodiments, the acid comprises formic acid, acetic acid, citric acid, propanoic acid, butyric acid, benzoic acid, phosphoric acid, hydrochloric acid, trifluoroacetic acid, sulfuric acid, or combinations thereof. In some embodiments, the acid comprises an organic acid. In some embodiments, the acid comprises formic acid, acetic acid, citric acid, propanoic acid, butyric acid, or benzoic acid. In some embodiments, the acid comprises acetic acid.

In some embodiments, the method of preparing a compound of Formula (II-a) or Formula (II-b) further comprises preparing the compound of Formula (V), the method comprising: (a1) forming a third reaction mixture comprising a compound of Formula (III):

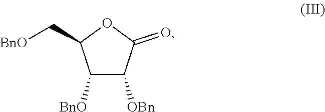 [SMILES: O=C1O[C@H](COCc2ccccc2)[C@@H](OCc2ccccc2)[C@H]1OCc1ccccc1]

an amine of the formula H—R^{a}; and a third base that is R³MgX³ or R³Li; wherein R³ is methyl, ethyl, n-propyl, isopropyl, n-butyl, tert-butyl, or phenyl; and X³ is Cl, Br, or I;
thereby providing the compound of Formula (V).

In some embodiments, the amine has the formula

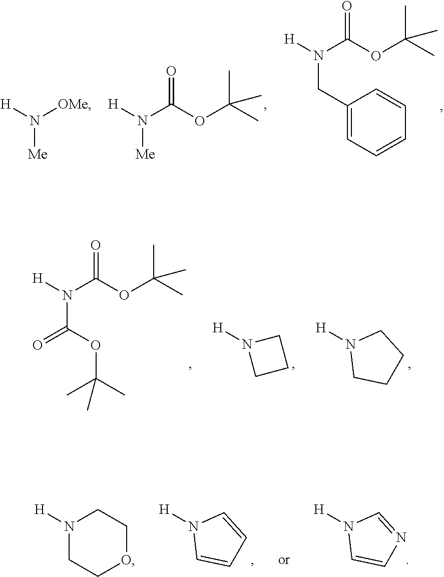

In some embodiments, the amine has the formula

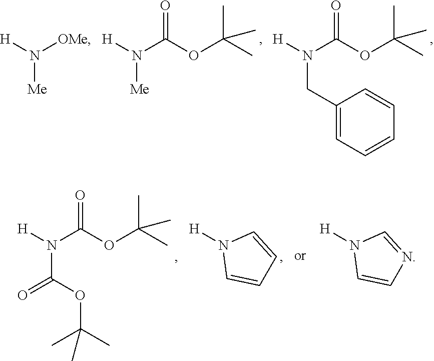

In some embodiments, the amine has the formula

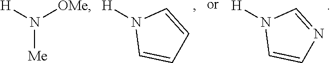

In some embodiments, the amine has the formula

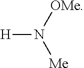

In some embodiments, X³ is Cl, Br, or I. In some embodiments, X³ is Br or I. In some embodiments, X³ is Cl. In some embodiments, X³ is Br. In some embodiments, X³ is I.

In some embodiments, the third base is R³MgX^{3,} wherein R³ is methyl, ethyl, n-propyl, isopropyl, n-butyl, tert-butyl, or phenyl; and X³ is Cl, Br, or I. In some embodiments, the third base is R³MgX³, wherein R³ is methyl, isopropyl, n-butyl, tert-butyl, or phenyl; and X³ is Cl or Br. In some embodiments, the third base is R³MgCl, wherein R³ is methyl, isopropyl, tert-butyl, or phenyl. In some embodiments, the third base is MeMgCl, iPrMgCl, or t-BuMgCl. In some embodiments, the third base is iPrMgCl.

In some embodiments, the amine has the formula

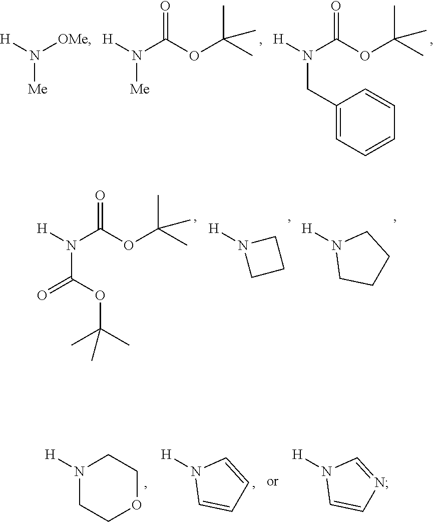

and
the third base is iPrMgCl.
   In some embodiments, the amine has the formula

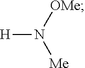

and
the third base is iPrMgCl.

Any suitable solvent can be used in preparing the compound of Formula (V) in the method described herein. In some embodiments, the third reaction mixture further comprises a third solvent that is an ether solvent or a chlorinated solvent. In some embodiments, the third reaction mixture further comprises a third solvent that is tetrahydrofuran (THF), 2-methyltetrahydrofuran, methyl tert-butyl ether, cyclopentyl methyl ether, toluene, n-heptane, 1,2-dichloroethane, chloroform, or chlorobenzene, or a combination thereof. In some embodiments, the third reaction mixture further comprises a third solvent that is tetrahydrofuran (THF), 2-methyltetrahydrofuran, methyl tert-butyl ether, or a combination thereof. In some embodiments, the third solvent is tetrahydrofuran (THF).

Any suitable temperature can be used in preparing the compound of Formula (V). In some embodiments, the third reaction mixture is maintained at a temperature of from about −78° C. to about 40° C. In some embodiments, the third reaction mixture is maintained at a temperature of from about −20° C. to about 25° C. In some embodiments, the third reaction mixture is maintained at a temperature of from about 0° C. to about 25° C. In some embodiments, the third reaction mixture is maintained at a temperature of from about 10° C. to about 25° C. In some embodiments, the third reaction mixture is maintained at a temperature of from about 15° C. to about 25° C. In some embodiments, the third reaction mixture is maintained at a temperature of about 20° C.

In some embodiments, the method comprises: (a1) forming the third reaction mixture comprising the compound of Formula (III) having the structure:

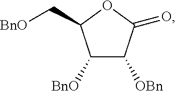 [SMILES: O=C1O[C@H](COCc2ccccc2)[C@@H](OCc2ccccc2)[C@H]1OCc1ccccc1]

the amine having the formula

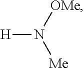

and
iPrMgCl, thereby forming the compound of Formula (V) having the structure:

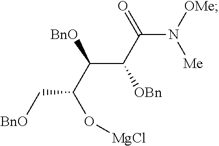

(a) preparing the first input mixture in the first reactor, wherein the first input mixture comprises TMS-Cl, PhMgCl, iPrMgCl, and the compound of Formula (IV) having the structure:

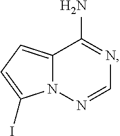 [SMILES: Nc1ncnn2c(I)ccc12]

wherein the first reactor provides the first output mixture; and
(b) adding the first output mixture and the compound of Formula (V) to the second reactor, thereby forming the compound of Formula (II-a) or Formula (II-b) having the structure:

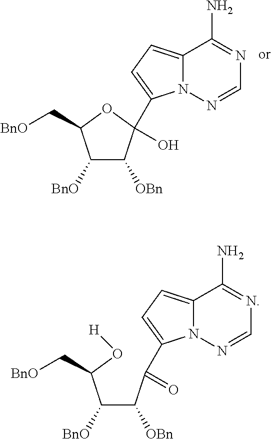 [SMILES: Nc1ncnn2c(C3(O)O[C@H](COCc4ccccc4)[C@@H](OCc4ccccc4)[C@H]3OCc3ccccc3)ccc12 ; "or" ; Nc1ncnn2c(C(=O)[C@H](OCc3ccccc3)[C@H](OCc3ccccc3)[C@H](O)COCc3ccccc3)ccc12]

The compound of Formula (II-a) having the structure:

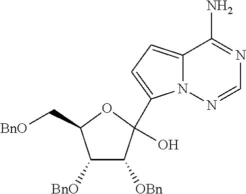 [SMILES: Nc1ncnn2c(C3(O)O[C@H](COCc4ccccc4)[C@@H](OCc4ccccc4)[C@H]3OCc3ccccc3)ccc12]

is also known as (3R,4R,5R)-2-(4-aminopyrrolo[2,1-ƒ][1,2,4]triazin-7-yl)-3,4-bis(benzyloxy)-5-((benzyloxy)methyl)tetrahydrofuran-2-ol.

As is generally understood in the art, the compound of Formula (II-a) having the structure:

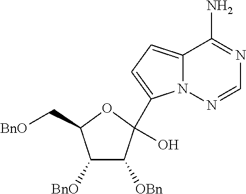 [SMILES: Nc1ncnn2c(C3(O)O[C@H](COCc4ccccc4)[C@@H](OCc4ccccc4)[C@H]3OCc3ccccc3)ccc12]

exists in an equilibrium with a compound of Formula (II-b) having the structure:

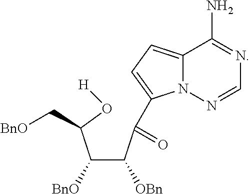 [SMILES: Nc1ncnn2c(C(=O)[C@H](OCc3ccccc3)[C@H](OCc3ccccc3)[C@H](O)COCc3ccccc3)ccc12]

Accordingly, as used herein, the compound of Formula (II-a) having the above structure when recited alone is understood to mean the compound of Formula (II-a) and/or the compound of Formula (II-b) or any combination of the two species.

The method of the present disclosure is amenable to synthesis of gram to kilogram quantities of the compound of Formula (II-a) or Formula (II-b) from the compound of Formula (III). In some embodiments, the third reaction mixture comprises at least 50 g, 100 g, 200 g, 300 g, 400 g, 500 g, 600 g, 700 g, 800 g, 900 g, 1 kg, 2 kg, 3 kg, 4 kg, 5 kg, 10 kg, 20 kg, 30 kg, 40 kg, 50 kg, 100 kg, 200 kg, 500 kg, or at least 1000 kg or more of the compound of Formula (III). In some embodiments, the third reaction mixture comprises at least 1 kg of the compound of Formula (III). In some embodiments, the third reaction mixture comprises from about 50 g to about 100 kg, e.g., from about 50 g to about 20 kg, or from about 30 g to about 20 kg, of the compound of Formula (III). In some embodiments, the third reaction mixture comprises from about 5 kg to about 15 kg of the compound of Formula (III). For example, in some embodiments, the third reaction mixture comprises about 10 kg of the compound of Formula (III).

The compound of Formula (III) having the structure:

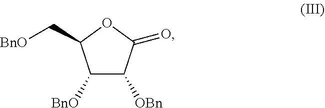 [SMILES: O=C1O[C@H](COCc2ccccc2)[C@@H](OCc2ccccc2)[C@H]1OCc1ccccc1]

is also known as (3R,4R,5R)-3,4-bis(benzyloxy)-5-((benzyloxy)methyl)dihydrofuran-2(3H)-one.

The compound of Formula (IV) having the structure:

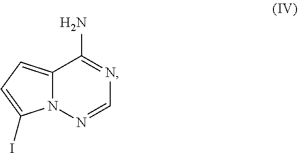 [SMILES: Nc1ncnn2c(I)ccc12]

is also known as 7-iodopyrrolo[2,1-ƒ][1,2,4]triazin-4-amine.

The methods of the present disclosure can provide the compound of Formula (II-a) or Formula (II-b) from the compound of Formula (III) or the compound of Formula (V) in any suitable yield. For example, the compound of Formula (II-a) or Formula (II-b) can be prepared in a yield of at least 50%, 55%, 60%, 65%, 70%, 75%, 80%, 85%, 90%, 95%, 97%, 98%, or at least 99%. In some embodiments, the yield of Formula (II-a) or Formula (II-b) is from about 60% to about 100%. In some embodiments, the yield of Formula (II-a) or Formula (II-b) is from about 70% to about 80% or from about 75% to about 85%. In some embodiments, the yield of Formula (II-a) or Formula (II-b) is about 60%, about 70%, about 72%, about 74%, about 75%, about 76%, about 78%, about 80%, about 82%, about 84%, about 85%, about 86%, about 88%, about 90%, about 95%, about 97%, about 98%, or about 99%. In some embodiments, the yield of Formula (II-a) or Formula (II-b) is about 79%. In some embodiments, the yield of Formula (II-a) or Formula (II-b) is from about 60% to about 90%. In some embodiments, the yield of Formula (II-a) or Formula (II-b) is from about 70% to about 90%. In some embodiments, the yield of Formula (II-a) or Formula (II-b) is from about 70% to about 80%. In some embodiments, the yield of Formula (II-a) or Formula (II-b) is from about 75% to about 85%.

The methods of the present disclosure can provide the compound of Formula (II-a) or Formula (II-b) from the compound of Formula (III) or the compound of Formula (V) in any suitable purity. For example, the compound of Formula (II-a) or Formula (II-b) can be prepared in a purity of from about 90% to about 100%, such as from about 95% to about 100% or from about 98% to about 100%. In some embodiments, the purity of the compound of Formula (II-a) or Formula (II-b) is from about 98% to about 100%. In some embodiments, the compound of Formula (II-a) or Formula (II-b) is prepared in a purity of about 90%, about 95%, about 96%, about 97%, about 98%, about 99%, about 99.9%, about 99.99%, about 99.999%, about 99.9999%, or about 99.99999%. In some embodiments, the compound of Formula (II-a) or Formula (II-b) is prepared in a purity of about 99.92%. In some embodiments, the compound of Formula (II-a) or Formula (II-b) is prepared in a purity of from about 95% to about 99.999%, from about 98% to about 99.999%, from about 98% to about 99.99%, or from about 99% to about 99.99%. In some embodiments, the purity of the compound of Formula (II-a) or Formula (II-b) is from about 90% to about 100%.

### IV. EXAMPLES

### Example 1. Synthesis of (3R,4R,5R)-2-(4-aminopyrrolo[2,1-ƒ][1,2,4]triazin-7-yl)-3,4-bis(benzyloxy)-5-((benzyloxy)methyl)tetrahydrofuran-2-ol

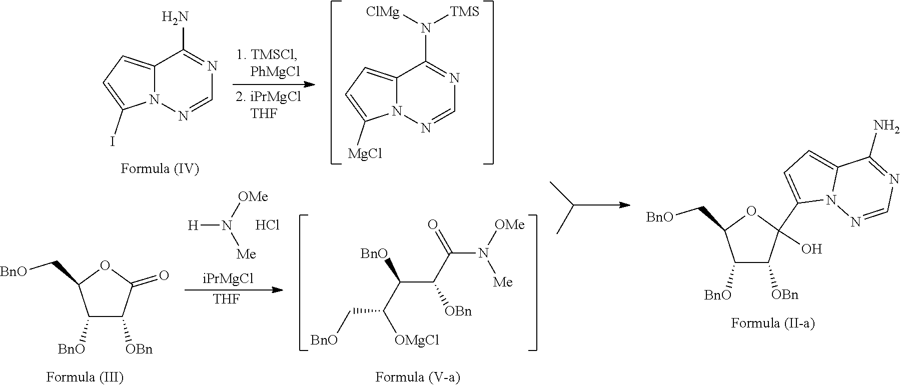

A reactor, under nitrogen gas at atmospheric pressure, was charged with the compound of Formula (IV) (1.2 equiv) and tetrahydrofuran (5.6 volumes). The contents were cooled to about −5° C. and trimethylsilyl chloride (2.4 equiv) was charged. After about 30 minutes agitation, the contents were cooled to about −10° C. and phenylmagnesium chloride (2.4 equiv) was added. The contents were then agitated for about 30 min at about −10° C. before adjusting to about −20° C. Isopropylmagnesium chloride (1.2 equiv) was added. The contents were adjusted to about −20° C. and agitated for about 1 hour. A second reactor was charged with the compound of Formula (III) (1.0 equiv, scaling factor), N,O-dimethylhydroxylamine·HCl (1.1 equiv) and tetrahydrofuran (5.6 volumes). The contents were cooled to about −20° C. and isopropylmagnesium chloride (2.25 equiv) was added. The contents were adjusted to about 20° C. and agitated for about 30 minutes. The contents from the two reactors were combined and then rinsed forward with tetrahydrofuran (1.7 volumes). The mixture was agitated for about 8 hours at about 20° C. A solution of acetic acid (0.95 volumes) in water (6 volumes) was added followed by toluene (3.8 volumes) and the mixture was agitated at about 20° C. for about 30 minutes. The layers were separated (aqueous discarded) and the organic layer was washed with a solution of 10 wt % potassium bicarbonate (5 volumes) and then three times with a solution of 10 wt % sodium chloride (5 volumes). The organic layer was concentrated under vacuum to about 5 volumes. Toluene (10 volumes) was charged and the concentration repeated. The contents were then polish filtered, rinsed forward with toluene (1.5 volumes) and concentrated under vacuum to 3 volumes. Methyl tert-butyl ether (7.4 volumes) was added followed by seed crystals of the compound of Formula (II-a) (0.001×) and agitated at about 22° C. about 1 hour. n-Heptane (4.4 volumes) was then added over about 1 hour, the contents were adjusted to about 0° C. over about 3 hours and then the mixture was agitated for about 12 hours at about 0° C. The slurry was filtered and the cake was rinsed with n-heptane (0.4 volumes) and methyl tert-butyl ether (1.5 volumes), then dried under vacuum to provide the compound of Formula (II-a).

Although the foregoing disclosure has been described in some detail by way of illustration and Example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

What is claimed is:

1. A method of preparing a compound of Formula (II-a) or Formula (II-b): 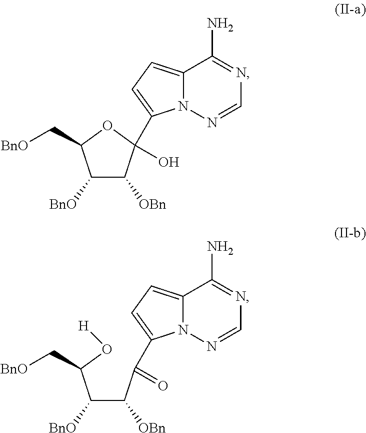 [SMILES: Nc1ncnn2c(C3(O)O[C@H](COCc4ccccc4)[C@@H](OCc4ccccc4)[C@H]3OCc3ccccc3)ccc12 ; Nc1ncnn2c(C(=O)[C@H](OCc3ccccc3)[C@H](OCc3ccccc3)[C@H](O)COCc3ccccc3)ccc12]
comprising:
(a) preparing a first input mixture, wherein the first input mixture comprises an amine protecting agent, a first base, a metalating agent, and a compound of Formula (IV): 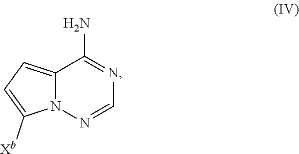
to provide a first output mixture; and
(b) preparing a second input mixture comprising the first output mixture and a compound of Formula (V) to provide a second output mixture comprising the compound of Formula (II-a) or Formula (II-b), wherein the compound of Formula (V) has the structure: 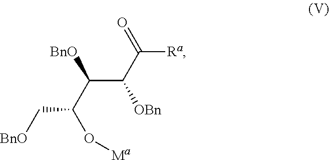
wherein
R^{a} is 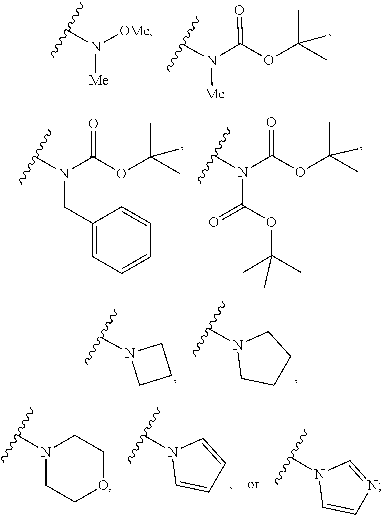
M^{a} is Li or MgX^{a};
X^{a} is Cl, Br, or I; and
X^{b} is Cl, Br, or I.

2. The method of claim 1, for preparing the compound of Formula (II-a) or Formula II-b): 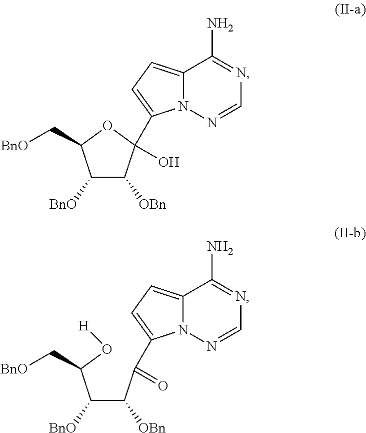 [SMILES: Nc1ncnn2c(C3(O)O[C@H](COCc4ccccc4)[C@@H](OCc4ccccc4)[C@H]3OCc3ccccc3)ccc12 ; Nc1ncnn2c(C(=O)[C@H](OCc3ccccc3)[C@H](OCc3ccccc3)[C@H](O)COCc3ccccc3)ccc12]
comprising:
(a) preparing the first input mixture in a first reactor, wherein the first input mixture comprises an amine protecting agent, the first base, the metalating agent, and the compound of Formula (IV): 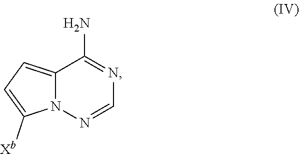
wherein the first reactor provides the first output mixture; and
(b) adding the first output mixture and the compound of Formula (V) to form the second input mixture in a second reactor, wherein the compound of Formula (V) has the structure: 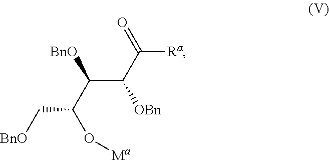
wherein
R^{a} is 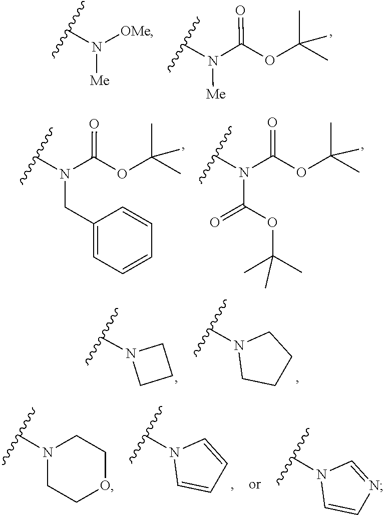
M^{a} is Li or MgX^{a};
X^{a} is Cl, Br, or I;
X^{b} is Cl, Br, or I; and
the second reactor provides the second output mixture comprising the compound of Formula (II-a) or Formula (II-b).

3. The method of claim 2, for preparing the compound of Formula (II-a) or Formula II-b): 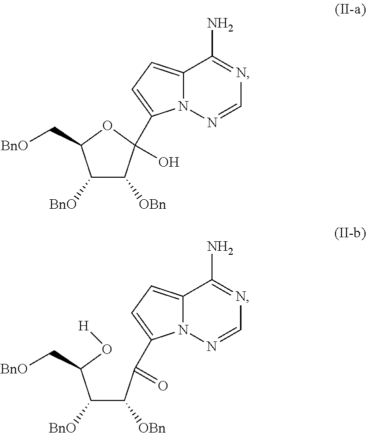 [SMILES: Nc1ncnn2c(C3(O)O[C@H](COCc4ccccc4)[C@@H](OCc4ccccc4)[C@H]3OCc3ccccc3)ccc12 ; Nc1ncnn2c(C(=O)[C@H](OCc3ccccc3)[C@H](OCc3ccccc3)[C@H](O)COCc3ccccc3)ccc12]
comprising:
(a) preparing the first input mixture in a first reactor, wherein the first input mixture comprises an amine protecting agent, the first base, the metalating agent, and the compound of Formula (IV): 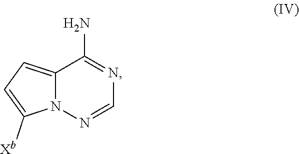
wherein the first reactor provides the first output mixture; and
(b) adding the first output mixture and the compound of Formula (V) to form the second input mixture in a second reactor, wherein the compound of Formula (V) has the structure: 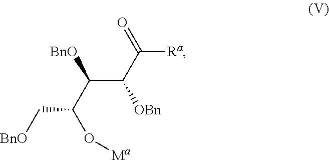
wherein
R^{a} is 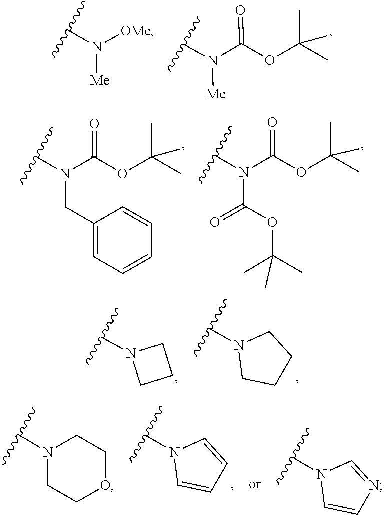
M^{a} is Li or MgX^{a};
X^{a} is Cl, Br, or I;
X^{b} is Cl, Br, or I;
the first base is R¹MgX¹ or R¹Li;
R¹ is methyl, ethyl, n-propyl, isopropyl, n-butyl, tert-butyl, or phenyl;
X¹ is Cl, Br, or I; and
the second reactor provides the second output mixture comprising the compound of Formula (II-a) or Formula (II-b),
with the proviso that when R^{a} is 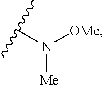 [SMILES: *N(C)OC]
M^{a} is MgCl, and R¹ is methyl, then X¹ is Cl or I.

4. The method of claim 2, for preparing the compound of Formula (II-a) or Formula II-b): 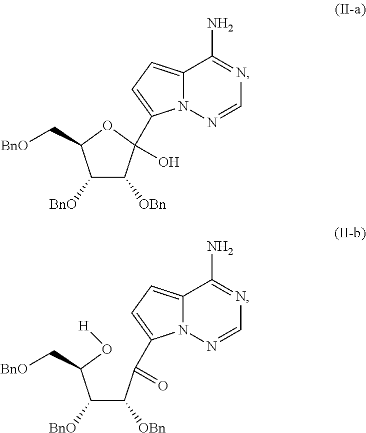 [SMILES: Nc1ncnn2c(C3(O)O[C@H](COCc4ccccc4)[C@@H](OCc4ccccc4)[C@H]3OCc3ccccc3)ccc12 ; Nc1ncnn2c(C(=O)[C@H](OCc3ccccc3)[C@H](OCc3ccccc3)[C@H](O)COCc3ccccc3)ccc12]
comprising:
(a) preparing the first input mixture in the first reactor, wherein the first input mixture comprises an amine protecting agent, the first base, the metalating agent, and the compound of Formula (IV): 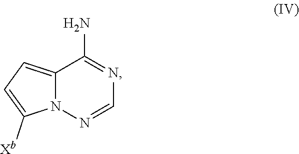
wherein the first reactor provides the first output mixture; and
(b) adding the first output mixture and the compound of Formula (V) to form the second input mixture in a second reactor, wherein the compound of Formula (V) has the structure: 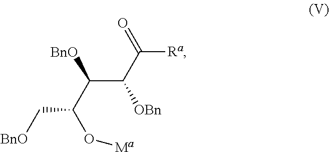
wherein
R^{a} is 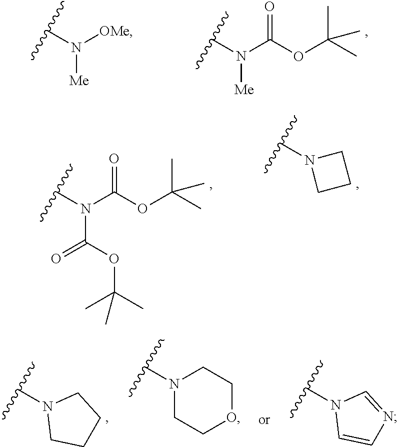
M^{a} is Li or MgX^{a};
X^{a} is Cl, Br, or I;
X^{b} is Cl, Br, or I;
the metalating agent is R²MgX² or R²Li;
R² is methyl, ethyl, n-propyl, isopropyl, n-butyl, tert-butyl, or phenyl;
X² is Cl, Br, or I; and
the second reactor provides the second output mixture comprising the compound of Formula (II-a) or Formula (II-b).

5. The method of claim 2, for preparing the compound of Formula (II-a) or Formula II-b): 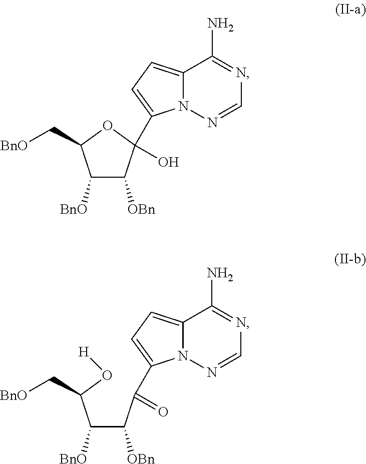 [SMILES: Nc1ncnn2c(C3(O)O[C@H](COCc4ccccc4)[C@@H](OCc4ccccc4)[C@H]3OCc3ccccc3)ccc12 ; Nc1ncnn2c(C(=O)[C@H](OCc3ccccc3)[C@H](OCc3ccccc3)[C@H](O)COCc3ccccc3)ccc12]
comprising:
(a) preparing the first input mixture in the first reactor, wherein the first input mixture comprises an amine protecting agent, the first base, the metalating agent, and the compound of Formula (IV): 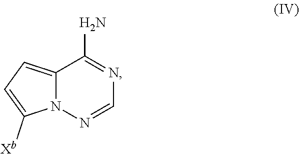
wherein the first reactor provides the first output mixture; and
(b) adding the first output mixture and the compound of Formula (V) to form the second input mixture in a second reactor, wherein the compound of Formula (V) has the structure: 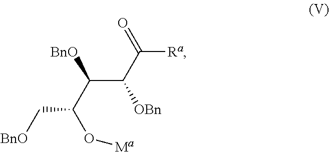
wherein
R^{a} is 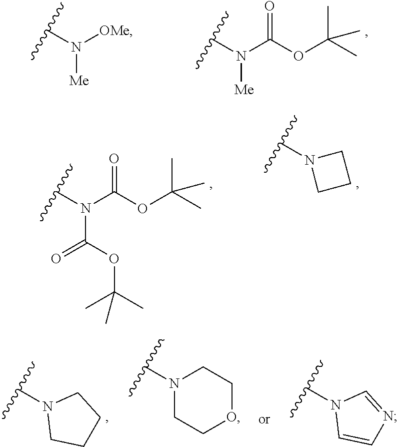
M^{a} is Li or MgX^{a};
X^{a} is Cl, Br, or I;
X^{b} is Cl, Br, or I;
the first base is R¹MgX¹ or R¹Li;
R¹ is methyl, ethyl, n-propyl, isopropyl, n-butyl, tert-butyl, or phenyl;
X¹ is Cl, Br, or I;
the metalating agent is R²MgX² or R²Li;
R² is methyl, ethyl, n-propyl, isopropyl, n-butyl, tert-butyl, or phenyl;
X² is Cl, Br, or I; and
the second reactor provides the second output mixture comprising the compound of Formula (II-a) or Formula (II-b),
with the proviso that when R^{a} is 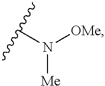 [SMILES: *N(C)OC]
M^{a} is MgCl, and R¹ is methyl, then X¹ is Cl or I.

6. The method of any one of claims 1 to 5, wherein the amine protecting agent is trifluoroacetic anhydride, di(tert-butyl) dicarbonate, trimethylsilyl chloride (TMSCl), triethylsilyl chloride (TESCl), triisopropylsilyl chloride, tert-butyldimethylsilyl chloride (TBDMSCl), tert-butyldiphenylsilyl chloride (TBDPSCl), triphenylsilyl chloride, or 1,2-bis(chlorodimethylsilyl)ethane.

7. The method of any one of claims 1 to 6, wherein
the first base is R¹MgX¹ or R¹Li;
R¹ is methyl, ethyl, n-propyl, isopropyl, n-butyl, tert-butyl, or phenyl; and
X¹ is Cl, Br, or I.

8. The method of any one of claims 1 to 6, wherein
the first base is R¹MgX¹ or R¹Li;
R¹ is methyl, ethyl, n-propyl, isopropyl, n-butyl, tert-butyl, or phenyl; and
X¹ is Cl, Br, or I,
with the proviso that when R¹ is methyl, then X¹ is Cl or I.

9. The method of any one of claims 1 to 6, wherein
the first base is R¹MgX¹ or R¹Li;
R¹ is ethyl, n-propyl, isopropyl, n-butyl, tert-butyl, or phenyl; and
X¹ is Cl, Br, or I.

10. The method of any one of claims 1 to 6, wherein
the first base is R¹MgX¹ or R¹Li;
R¹ is methyl, ethyl, n-propyl, isopropyl, n-butyl, tert-butyl, or phenyl; and
X¹ is Cl or I.

11. The method of any one of claims 1 to 6, wherein
the first base is R¹MgX¹ or R¹Li;
R¹ is ethyl, n-propyl, isopropyl, n-butyl, tert-butyl, or phenyl; and
X¹ is Cl or I.

12. The method of any one of claims 1 to 11, wherein the first base is PhMgCl.

13. The method of any one of claims 1 to 12, wherein
the metalating agent is R²MgX² or R²Li;
R² is methyl, ethyl, n-propyl, isopropyl, n-butyl, tert-butyl, or phenyl; and
X² is Cl, Br, or I.

14. The method of any one of claims 1 to 13, wherein the metalating agent is iPrMgCl.

15. The method of claim 1, wherein
the amine protecting agent is trimethylsilyl chloride (TMSCl);
the first base is PhMgCl;
the metalating agent is iPrMgCl; and
M^{a} is MgCl.

16. The method of any one of claims 1 to 15, wherein the first input mixture further comprises a first solvent that is tetrahydrofuran (THF), 2-methyltetrahydrofuran, methyl tert-butyl ether, cyclopentyl methyl ether, toluene, n-heptane, 1,2-dichloroethane, chloroform, or chlorobenzene, or a combination thereof.

17. The method of claim 16, wherein the first solvent is tetrahydrofuran (THF).

18. The method of any one of claims 2 to 17, wherein the first reactor is maintained at a temperature of from about −78° C. to about 20° C.

19. The method of any one of claims 2 to 18, wherein the first reactor is cooled to a temperature of from about −20° C. to about 0° C.

20. The method of any one of claims 1 to 19, wherein the second input mixture further comprises a second solvent that is tetrahydrofuran (THF), 2-methyltetrahydrofuran, methyl tert-butyl ether, cyclopentyl methyl ether, toluene, n-heptane, 1,2-dichloroethane, chloroform, or chlorobenzene, or a combination thereof.

21. The method of claim 20, wherein the second solvent is tetrahydrofuran (THF).

22. The method of any one of claims 2 to 21, wherein the second reactor is maintained at a temperature of from about −20° C. to about 40° C.

23. The method of any one of claims 2 to 22, wherein the second reactor is maintained at a temperature of from about 10° C. to about 30° C.

24. The method of any one of claims 2 to 23, wherein the second reactor is maintained at a temperature of about 20° C.

25. The method of any one of claims 1 to 24, further comprising combining the second output mixture and an acid.

26. The method of claim 25, wherein the acid comprises an organic acid or a mineral acid, or combinations thereof.

27. The method of claim 25 or 26, wherein the acid comprises formic acid, acetic acid, citric acid, propanoic acid, butyric acid, benzoic acid, phosphoric acid, hydrochloric acid, trifluoroacetic acid, sulfuric acid, or combinations thereof.

28. The method of any one of claims 25 to 27, wherein the acid comprises an organic acid.

29. The method of any one of claims 25 to 28, wherein the acid comprises formic acid, acetic acid, citric acid, propanoic acid, butyric acid, or benzoic acid.

30. The method of any one of claims 25 to 29, wherein the acid comprises acetic acid.

31. The method of any one of claims 1 to 30, further comprising preparing the compound of Formula (V), the method comprising:
(a1) forming a third reaction mixture comprising a compound of Formula (III): 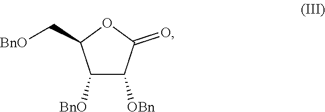 [SMILES: O=C1O[C@H](COCc2ccccc2)[C@@H](OCc2ccccc2)[C@H]1OCc1ccccc1]
an amine of the formula H—R^{a}; and
a third base that is R³MgX³ or R³Li;
wherein
R³ is methyl, ethyl, n-propyl, isopropyl, n-butyl, tert-butyl, or phenyl; and
X³ is Cl, Br, or I;
thereby providing the compound of Formula (V).

32. The method of claim 31, wherein the amine has the formula

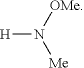

33. The method of claim 31 or 32, wherein
the amine has the formula 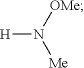
and
the third base is iPrMgCl.

34. The method of any one of claims 31 to 33, wherein the third reaction mixture further comprises a third solvent that is tetrahydrofuran (THF), 2-methyltetrahydrofuran, methyl tert-butyl ether, cyclopentyl methyl ether, toluene, n-heptane, 1,2-dichloroethane, chloroform, or chlorobenzene, or a combination thereof.

35. The method of claim 34, wherein the third solvent is tetrahydrofuran (THF).

36. The method of any one of claims 31 to 35, wherein the third reaction mixture is maintained at a temperature of from about −78° C. to about 40° C.

37. The method of any one of claims 31 to 36, wherein the third reaction mixture is maintained at a temperature of from about −20° C. to about 25° C.

38. The method of any one of claims 2 to 37, wherein the method comprises:
(a1) forming the third reaction mixture comprising the compound of Formula (III) having the structure: 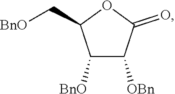 [SMILES: O=C1O[C@H](COCc2ccccc2)[C@@H](OCc2ccccc2)[C@H]1OCc1ccccc1]
the amine having the formula 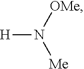
and
iPrMgCl, thereby forming the compound of Formula (V) having the structure: 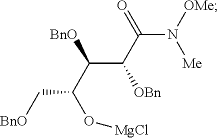
(a) adding the first input mixture to the first reactor, wherein the first input mixture comprises TMS-Cl, PhMgCl, iPrMgCl, and the compound of Formula (IV) having the structure: 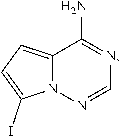 [SMILES: Nc1ncnn2c(I)ccc12]
wherein the first reactor provides the first output mixture; and
(b) adding the first output mixture and the compound of Formula (V) to the second reactor, thereby forming the compound of Formula (II-a) or Formula (II-b) having the structure:

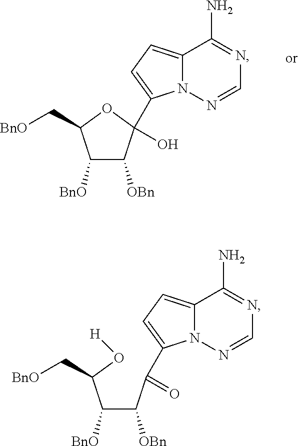 [SMILES: Nc1ncnn2c(C3(O)O[C@H](COCc4ccccc4)[C@@H](OCc4ccccc4)[C@H]3OCc3ccccc3)ccc12 ; Nc1ncnn2c(C(=O)[C@H](OCc3ccccc3)[C@H](OCc3ccccc3)[C@H](O)COCc3ccccc3)ccc12]

39. The method of any one of claims 1 to 38, wherein the yield of the compound of Formula (II-a) or Formula (II-b) is from about 60% to about 90%.

40. The method of any one of claims 1 to 39, wherein the purity of the compound of Formula (II-a) or Formula (II-b) is from about 90% to about 100%.

41. The method of any one of claims 2 to 40, wherein the first reactor and the second are different reactors

42. The method of any one of claims 2 to 41, wherein the first reactor and the second reactor are the same type of reactor.

43. The method of any one of claims 2 to 42, wherein the first reactor and the second reactor are different types of reactors.

44. The method of any one of claims 2 to 40, wherein the first reactor and the second reactor are a single reactor.

45. The method of claim 44, wherein the single reactor is a continuous flow reactor, a plug flow reactor, a continuous tubular reactor, or a mixed flow reactor.

46. The method of claim 44 or 45, wherein the first reactor is a first reaction zone in the single reactor and the second reactor is a second reaction zone in the single reactor.
